(19) United States
(12) Patent Application Publication
(10) Pub. No.: US 2020/0312455 A1
(43) Pub. Date: Oct. 1, 2020

(54) SYSTEMS AND METHODS FOR DETERMINING HEALTH CONDITION OF AN INDIVIDUAL

(71) Applicant: MILLIONEYES HEALTHCARE TECHNOLOGIES PRIVATE LIMITED, Bengaluru (IN)

(72) Inventor: Gaurav Bhalotia, Bengaluru (IN)

(73) Assignee: MILLIONEYES HEALTHCARE TECHNOLOGIES PRIVATE LIMITED, Bengaluru (IN)

(21) Appl. No.: 16/830,184

(22) Filed: Mar. 25, 2020

(30) Foreign Application Priority Data
Mar. 26, 2019 (IN) 201941011807

(51) Int. Cl. G16H 50/20; G16H 30/40; G16H 10/60; G16H 20/60; G16H 20/30; G16H 20/10; G06T 7/00

(52) U.S. Cl. CPC G16H 50/20; G16H 30/40; G16H 10/60; G16H 20/60; G06T 2207/30004; G16H 20/10; G06T 7/0012; G06T 2207/30204; G06T 2207/30201; G16H 20/30

(57) ABSTRACT

The present disclosure is directed towards a method (300, 400) for determining a health condition of an individual. The method (300, 400) includes receiving, by a processing unit (102), an image of visual markers of the individual and determining, by the processing unit (102), the health condition of the individual based on the image of visual markers of the individual. The determination of the health condition of the individual comprises determination of a constitution type of the individual.

### FIELD

The present disclosure relates generally to determining a health condition of an individual. More specifically, the present disclosure relates to determining health condition utilizing an image of virtual markers of the individual.

### BACKGROUND

The human body has a complex dynamic equilibrium of chemicals, enzymes, electrolytes, vitamins, minerals, etc. The equilibrium of the human body gets disturbed when it is subjected to one or more stressors. The stressor may be in the form of a lifestyle, epidemic, occupational, environmental, etc. Recovery may be aided by a change in diet or lifestyle, or by medications. When this equilibrium is disturbed initially, the human body may feel sick. When this equilibrium is disturbed for a longer period, the human body may suffer from a disease. The human body has an internal mechanism to heal from such sickness/diseases and to re-attain natural equilibrium. Each individual person may have a different equilibrium and may have different resistance and reaction to stressors that may disturb the equilibrium. Therefore, each individual person may have different needs and requirements for sickness management.

### SUMMARY

This summary is provided to introduce concepts related to the present inventive subject matter. The summary is not intended to identify essential features of the claimed subject matter nor is it intended for use in determining or limiting the scope of the claimed subject matter. The embodiments described below are not intended to be exhaustive or to limit the disclosure to the precise forms disclosed in the following detailed description. Rather, the embodiments are chosen and described so that others skilled in the art may appreciate and understand the principles and practices of the present inventive subject matter.

In one aspect, the disclosure is directed towards a method for determining health condition of an individual. The method includes receiving, by a processing unit, an image of visual markers of the individual and determining, by the processing unit, the health condition of the individual based on the image of visual markers of the individual. The determination of the health condition of the individual comprises determination of a constitution type of the individual.

In another aspect, the disclosure is directed towards a computing system including a processing unit. The processing unit is configured to receive an image of visual markers of the individual and determine the health condition of the individual based on the image of visual markers of the individual. The determination of the health condition of the individual comprises determination of a constitution type of the individual.

Numerous advantages and benefits of the inventive subject matter disclosed herein will become apparent to those of ordinary skill in the art upon reading and understanding the present specification. It is to be understood, however, that the detailed description of the various embodiments and specific examples, while indicating preferred and/or other embodiments, are given by way of illustration and not limitation. Many changes and modifications within the scope of the present disclosure may be made without departing from the spirit thereof, and the disclosure includes all such modifications.

### BRIEF DESCRIPTION OF DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed disclosure and explain various principles and advantages of those embodiments.

FIG. 1 illustrates a block diagram of an exemplary computing system, in accordance with the embodiments of the present disclosure;

FIG. 2 illustrates a network environment for implementation of the computing system of FIG. 1, in accordance with the embodiments of the present disclosure;

FIG. 3 illustrates a method for determining a health condition of an individual based on an image of visual markers, in accordance with the embodiments of the present disclosure; and

FIG. 4 illustrates a method for determining a health condition of the individual based on the image of visual markers and other data, in accordance with the embodiments of the present disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

Hereinafter, the preferred embodiments of the present disclosure will be described in conjunction with the accompanying drawings, it should be understood that the preferred embodiments described herein are only used to illustrate and explain the present disclosure and are not intended to limit the present disclosure. While several examples are described in the description, modifications, adaptations, and other implementations are possible. Accordingly, the following detailed description is not limited by the disclosed examples.

References to “some embodiment”, “an embodiment”, “at least one embodiment”, “one example”, “an example”, “for example” and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in some embodiment” does not necessarily refer to the same embodiment.

The present disclosure relates to systems and methods for determining a health condition of an individual. The systems and the methods of the present disclosure may consider at least an image of visual markers of the individual and other details/data (hereinafter may be interchangeably referred to as profile or profile details/data) of the individual to determine health condition of the individual in a non-invasive manner.

In accordance with an example implementation of the present disclosure, details of an individual are received. The details may include at least an image of visual markers in a form of images of face, hair, nail, or other body parts of the individual. In accordance with some embodiments, the details of the individual may also include physiological parameters, medical history, previous medical records, previous test reports, lifestyle details, diet intake, water intake, stress level, occupational details, etc. The received image along with the other details are processed to determine health condition of the individual in a non-invasive manner.

The systems and methods of the present disclosure may maintain a database of diseases, constitution types of the human body, health fingerprints (hereinafter interchangeably referred to as health characteristics), and health recommendations. The database may include details for each type of diseases, constitution types of the human body, health fingerprints, health recommendations and their corresponding diagnostic parameters and/or indicators. The details of the individual are correlated with the diagnostic parameters and/or indicators included in the database to determine whether the individual is suffering from a disease and/or what is the constitution type of the body of the individual and/or what is the health fingerprint and/or what are the health recommendations for the individual to maintain or improve health.

With the systems and methods of the present disclosure, the health condition an individual may be determined by linking visuals (i.e. the image of virtual markers) to the underlying health of the human body and mind. As a result, an overall health determination may be done remotely, in a non-invasive manner, and without the use of any specific medical equipment.

With the systems and methods of the present disclosure, the health condition of the individual may be determined by extracting one or more diagnostic parameters from the image of visual markers of the individual and further by determining a correlation between the extracted one or more diagnostic parameters and a stored set of diagnostic parameters corresponding to a plurality of health conditions. Further, weightages are assigned to the determined correlation based on a strength of correlation and a determination of the health condition is done based on the determined weightages. In some embodiments, the health condition of the individual may further be determined or updated by extracting one or more diagnostic parameters from the profile data of the individual.

With the systems and methods of the present disclosure, the determined health condition may be indicative of a sickness or a disease, reactive allergies, symptoms of a disease, susceptibility to a disease, tendency to develop illness, a functional imbalance, and a prediction of a functional imbalance. The determined health condition of the individual is also utilized to identify root cause of a disease/sickness and its management by providing recommendations related to health supplements, medicines, medical support, exercise, diet management, lifestyle management, personalized body care products, wellness plan, precautions, additional health test, sickness management, and other type of medical support.

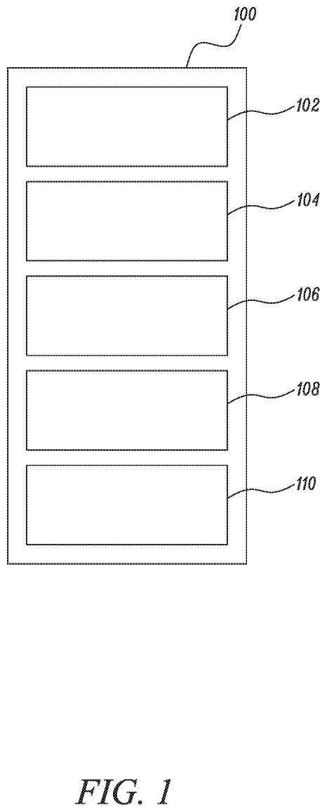

FIG. 1 illustrates a computing system 100 for determining a health condition of an individual, according to an embodiment of the present disclosure. The computing system 100 may include a processing unit 102, a memory 104, a storage 106, an input/output module 108, and a network interface 110.

The computing system 100 depicted in FIG. 1 may be implemented in any suitable computing environment, such as one or more of, a desktop or a laptop computer, a computer server, a handheld device, tablet, phablet, a mobile phone, a Personal Digital Assistant (PDA), a smart phone or a computer provided over a wired or wireless network. In addition, the computing system 100 may be divided into more systems than shown.

The processing unit 102 may include one or more microprocessors, microcomputers, microcontrollers, programmable logic controller, DSPs (digital signal processors), central processing units, state machines, logic circuitry, or any other device or devices that process/manipulate information or signals based on operational or programming instructions. The processing unit 102 may be implemented using one or more controller technologies, such as Application Specific Integrated Circuit (ASIC), Reduced Instruction Set Computing (RISC) technology, Complex Instruction Set Computing (CISC) technology, etc. The processing unit 102 may be configured to execute the instructions stored in the memory 104 to perform the predetermined operations. The processing unit 102 is also configured to receive or transmit data from other units, such as the input/output module 108 and the network interface 110, of the computing system 100. The memory 104 may be Random Access Memory (RAM) or Read Only Memory (ROM) and may be configured to store a set of instructions that are executable by the processing unit 102 to perform the predetermined operations.

The storage 106 may be a hard-disk drive or a removable storage drive, such as, a floppy-disk drive, optical-disk drive, and the like. The storage 106 may also be a means for loading computer programs or other instructions into the computing system 100. In an example implementation, the storage 106 may be provided outside the computing system 100 as a standalone device or as a part of a database server. When the storage 106 is provided outside the computing system 100, the computing system 100 may be coupled with the storage 106 through a wired or wireless communication.

The storage 106 may maintain a first database for disease types (hereinafter interchangeably referred as sickness types). The first database may include details of a plurality of disease types and one or more corresponding diagnostic parameters/indicators. In accordance with an embodiment, the diagnostic parameters/indicators represent evaluation factors that may be utilized to determine whether the individual is suffering from a particular type of disease or may be potentially at risk of contracting or developing a particular disease. In an exemplary embodiment, the first database may identify rheumatoid inflammation as a disease and the one or more corresponding diagnostic parameters/indicators corresponding to the rheumatoid inflammation may include skin markers on the face and/or forehead of the individual. In another exemplary embodiment, the first database may identify spleen disease and the one or more corresponding diagnostic parameters/indicators corresponding to the spleen disease may include skin coloration and/or specific patches on the face.

The storage 106 may further maintain a second database of health characteristics/fingerprints of the human body. The second database may include details of plurality of health characteristics/fingerprints and corresponding diagnostic parameters/indicators that may be utilized to determine the health characteristics of the individual. In accordance with various embodiments of the present disclosure, the health characteristics/fingerprint are indicative of pre-disposition of an individual at genotype and phenotype level. The pre-disposition of an individual represents strength of an individual towards various types of stressors. The strength may be defined in terms of immunity or may be the capability of the human body to retain equilibrium when subjected to various types of stressors. The strength indicator may therefore form basic characteristic of that individual or alternative may be referred as health fingerprint for that individual. The health fingerprint may be indicative of one or more medical conditions, reactive allergies, symptoms, tendencies to fall sick, or to develop illness.

The storage 106 may maintain a third database of constitution types of the human body. The third database may include details of a plurality of constitution types of the human body considered under various medicinal treatments and one or more corresponding diagnostic parameters/indicators. The corresponding diagnostic parameters/indicators represent an evaluation factor that may be utilized to categorize the individual under a specific constitution type. In an exemplary embodiment, the constitution type may be Vata, Pitta, and Kapha under the Ayurveda system of medicine. In another exemplary embodiment, the constitution type may be meridians under the traditional Chinese medicine. According to another exemplary embodiment, the constitution type may be as defined under the sasang Korean medicine. Further, the diagnostic parameters/indicators may include one or more of skin condition, facial condition, eye condition, hair texture, nail texture/color and so on.

The storage 106 may maintain a fourth database of recommendations. The fourth database may include information about the plurality of disease types, the plurality of constitution types, the plurality of health fingerprints, and the corresponding recommendations in respect of diet, exercise, precautions, etc., to improve individual's health or to maintain it.

Further, the storage 106 may maintain a fifth database (hereinafter interchangeably referred as patient database) for storing patient data/details. The patient database creates a profile for each patient which may include at least an image of visual markers, name, and age of the patient. In an example implementation, the profile may include comprehensive details about the patient, such as, name, address, age, height, weight, gender, current or previous images of face, hair, nail, or other body parts of the patient, physiological parameters, medical history, previous medical records, previous test reports, lifestyle details, diet intake, water intake, stress level, occupational details, and all such details that enables determination of disease type, constitution type, and health fingerprint associated with an individual. Therefore, the patient's profile may be created by providing a set of images, such as, face, eyes, hair, nails, or other body parts of the patient.

In an example implementation, the patient profile may also be created by providing a set of questionnaires in a digital form to the patient. Alternatively, the digital form is filled by an operator/user. The questionnaires may enable the patient to provide details related to name, address, age, height, weight, gender, physiological parameters, medical history, previous medical records, previous test reports, lifestyle details, diet intake, water intake, stress level, occupational details, etc. Once the questionnaire is completed, images of the patient may be recorded. The images of the patient may be utilized as visual markers that are indicative of present or future health traits of the patient. These visual markers result in early detection of various health conditions. Furthermore, the patient database may include details about a disease, a sickness, a constitution type, a health fingerprint, or one or more recommendations provided to the patient.

In an example implementation, a single database may be maintained in the storage 106 instead of having five different databases. The database(s) in the storage 106 is dynamic in nature, which may be created, updated, or modified with new details related to diseases, constitutions, health fingerprints, and recommendations. In an embodiment, the processing unit 102 may create, update, or modify the database(s) in the storage 106 based on new/updated details received from an operator/user of the computing system 100. In another embodiment, the processing unit 102 may create, update, or modify the database(s) in the storage 106 based on a new research or experimental details obtained by the processing unit 102 over a network or internet.

The processing unit 102 may be configured to extract a first set of diagnostic parameters, that may be required for determining a type of disease, from the image of the visual markers of the individual. In some embodiments, the processing unit 102 may be further configured to extract the first set of diagnostic parameters from the profile of the individual. The processing unit 102 may be further configured to determine a correlation between the extracted first set of diagnostic parameters and the diagnostic parameters/indicators associated with the plurality of disease types stored in the first database. In accordance with various embodiments, the processing unit 102 may be configured to employ Artificial Intelligence to determine a correlation between the extracted first set of diagnostic parameters and the diagnostic parameters/indicators associated with the plurality of disease types stored in the first database. The processing unit 102 may be further configured to assign weightages to the determined correlation based on a strength of the correlation and determine the disease type based on the assigned weightages.

In accordance with some embodiments, the Artificial Intelligence may be implemented by the processing unit 102 in the computing system 100 or by any other external device. The Artificial Intelligence may generate correlation between a disease type and corresponding skin condition or facial condition, or eye condition or hair texture or nail texture/color, if any. The correlation is a relationship between a disease type and a corresponding skin condition. The strength of relationship is being represented in the form of networks and weights. For example, corresponding parameters/indicators may include that a yellow color of eye or skin may indicate jaundice. Similarly, a specific pattern or a specific rash on skin portion may indicate fungal infection.

The processing unit 102 may be further configured to extract a second set of diagnostic parameters, that may be required for determining a type of health fingerprints, based on the image of the visual markers of the individual. In some embodiments, the processing unit 102 may be further configured to extract the second set of diagnostic parameters based on the profile of the individual. The processing unit 102 may be further configured to determine a correlation between the extracted second set of diagnostic parameters and the diagnostic parameters/indicators associated with the plurality of health fingerprints stored in the second database. In accordance with various embodiments, the processing unit 102 may be configured to employ the Artificial Intelligence to determine a correlation between the extracted second set of diagnostic parameters and the diagnostic parameters/indicators associated with the plurality of health fingerprints stored in the second database. The Artificial Intelligence may generate a correlation between a health fingerprint and corresponding skin condition or facial condition, or eye condition or hair texture or nail texture/color, or profile data if any. The correlation is a relationship between a health fingerprint and corresponding skin condition and profile data. The strength of relationship is being represented in the form of networks and weights. The processing unit 102 may be further configured to assign weightages to the determined correlation based on a strength of the correlation and determine the health fingerprint based on the assigned weightages.

In some embodiments, the processing unit 102 may be configured to determine a genotype, a phenotype, reactive allergy, symptoms of a disease, susceptibility to a disease, tendency to develop illness, a functional imbalance, a prediction of a functional imbalance and a root cause of a disease, as a part of health fingerprint determination. In an exemplary embodiment, the processing unit 102 may be configured to implement deep learning to identify characteristics of an individual based on the profile data of the individual. The computing system 100 may be further configured to analyze the visual markers of the individual to identify genotype and phenotype. Thereafter, the computing system 100 may be configured to determine health fingerprint for that individual based on the identified characteristics, genotype, and phenotype. The Artificial Intelligence may also establish a correlation between the visual markers of the individual and genotype/phenotype.

In another exemplary embodiment, the processing unit 102 may be configured to determine a root cause for any sickness of an individual based on the corresponding diagnostic parameters/indicators in the second database. The root causes may be based on quality of sleep/relaxation, exercise/movement, nutrition/hydration, stress/resilience, relationships, trauma, pathogens, and the environment. Based on the root cause, the health fingerprint of an individual may be determined by the processing unit 102. Any imbalance in the body due to the root cause may present a weak spot in the health fingerprint of the individual.

In yet another example, the processing unit 102 may be configured to determine that an individual has a tendency of being allergic to certain environmental conditions or food in the health fingerprint determination. In another example, the processing unit 102 may be configured to determine that an individual has slow metabolism and may be susceptible of being obese and may in future lead to diseases related to obesity in the health fingerprint determination.

In further embodiments, the processing unit 102 may be configured to determine or predict functional imbalance or disturbances in an individual when the individual is subjected to screening for symptoms, in the health fingerprint determination. The functional imbalance or disturbances may be categorized as Assimilation (Appetite and digestion), Elimination (sweat, stool, urine, toxicity, etc.), Circulation (Blood and heart), Immunity, Structural Integrity (cellular membrane, muscular system, and skeletal system), Metabolism (Energy regulator, transformation process, and Endocrine health), Communication and movement (Neurotransmitter, immune messengers, cognition, bodily movements, Regulatory decisions), and Tissue health (Plasma, Blood, Muscles, Fat, Bone/cartilage, Neurons/Marrow, and Reproductive tissue). The aggregation of functional imbalance and disturbance may result in signs or symptoms for one or more diseases at organ level. For example, the organ level disease may pertain to neurology, gastroenterology, endocrinology, cardiology, pulmonary, urology, hepatology, immunology, etc.

The processing unit 102 may be further configured to extract a third set of diagnostic parameters, that may be required for determining a type of constitution, based on the image of the visual markers of the individual. In some embodiments, the processing unit 102 may be further configured to extract the third set of diagnostic parameters based on the profile of the individual. The processing unit 102 may be further configured to determine a correlation between the extracted third set of diagnostic parameters and the diagnostic parameters/indicators associated with the plurality of constitution types stored in the third database. In accordance with various embodiments, the processing unit 102 may be configured to employ the Artificial Intelligence to determine the correlation between the extracted third set of diagnostic parameters and the diagnostic parameters/indicators associated with the plurality of constitution types stored in the third database. The Artificial Intelligence may generate a correlation between a constitution type and corresponding skin condition or facial condition, or eye condition or hair texture or nail texture/color, if any. The correlation is a relationship between a constitution type and corresponding skin condition or profile data. The strength of relationship is being represented in the form of networks and weights. The processing unit 102 may be further configured to assign weightages to the determined correlation based on the strength of correlation and determine the constitution type based on the assigned weightages.

The processing unit 102 may be further configured to identify correlation between the determined disease type, determined health fingerprint, and the determined constitution type to determine health condition of an individual. The processing unit 102 may be further configured to provide health recommendations to an individual based on the determined health condition. In accordance with some embodiments, the processing unit 102 may be configured to employ the Artificial Intelligence to identify correlation between the determined disease type, the determined health fingerprint, the determined constitution type, and the health recommendations based on the identified correlations. The Artificial Intelligence may implement deep learning to establish link/correlation between the determined different types of health conditions and their corresponding recommendations.

In an embodiment, the processing unit 102 may utilize the first, second, and third databases to determine health condition of the individual and accordingly may provide a health score. The determined health condition or the health score may serve as a seed for personalized recommendations. The processing unit 102 may be configured to utilize the fourth database to provide lifestyle or sickness management recommendations based on the determined health conditions or the health score of the individual. In an example implementation, the processing unit 102 may provide recommendation of supplements to provide health support, additional test(s) for further confirmation or starting of treatment plan, wellness plans designed for specific individual needs.

The processing unit 102 may be configured to process at least an image to determine different patterns/colors/shades/texture of eyes, hair, nails, or the skin of face or other body parts. These determined patterns/colors/shades/textures are linked with the diagnostic parameters/indicators in the first, second, and third databases to determine the disease type, the constitution type, and the health fingerprint associated with an individual. The linking is based on the correlations as established by the Artificial Intelligence. In another embodiment, the processing unit 102 may process the at least one image, as mentioned previously, along with the other details provided in the profile of the patient.

The processing unit 102 may be configured to determine correlation between the visual markers extracted from the at least one image and underlying health conditions to determine the state of health. The correlation between the visual markers and the underlying health conditions may be improved with time when further data related to any of the diseases, diagnosis, constitution, health fingerprint, recommendation, and patient is added to the database(s). Additionally, the correlation is further improved when other details of the patient from the patient profile is used in conjunction with the visual indicators/markers.

The processing unit 102 may further be configured to create and maintain a mapping in the storage 106 of all the diagnostic parameters/indicators corresponding to the diseases, constitution types, health fingerprint, and recommendations with respect to health conditions. The processing unit 102 may be configured to utilize this mapping to generate correlation between details provided in the patient's profile and the underlying health condition. The mapping may be updated by the processing unit 102 when there is any update or modification in the database(s).

The input/output module 108 (hereinafter referred to as I/O module 108) provides interface for input devices, for example, a keyboard, a keypad, a touchpad, a scanner, a mouse, a gesture, a touch sensor, a camera, an imaging system, a memory card reader or any other input device configured to receive inputs from the user. Further, I/O module 108 provides interface for output devices, for example, a display. The input and output devices communicate with the processing unit 102 through the I/O module 108.

The network interface 110 allows the computing system 100 to communicate with other network or remote devices through wired or wireless communication channel.

In accordance with an example implementation of the present subject matter, the computing system 100 may have imaging system (not shown in FIG. 1) as an integral part to capture images of an individual. In an example implementation, the imaging system may be provided external to the computing system 100. When the imaging system is provided external to the computing system 100, the imaging system provides one or more images of the individual to the computing system 100 over wired or wireless channel through the network interface 110 or though the I/O module 108.

The imaging system may be a camera, a webcam, a digital camera, an inbuilt camera of a portable device, a single lens reflection (SLR) camera, a digital SLR camera, or a point to shoot camera. The imaging system may have a functionality to store the one or more images on a storage device or to transfer wired/wirelessly to the computing system 100 directly or via network. The storage device of the imaging system may be provided to the I/O module 108 of the computing system 100 to read the one or more images from the storage device.

Referring to FIG. 1, an individual may directly operate the computing system 100 to provide required inputs to the computing system 100 and determine disease type, constitution type, and health fingerprint associated with an individual. In an example implementation, an operator, such as a doctor, an assistant, and any healthcare professional, may operate the computing system 100 to provide required inputs to the computing system 100 and determine the health condition. The computing system 100 may assist the doctors or healthcare professional for early detection of any health condition.

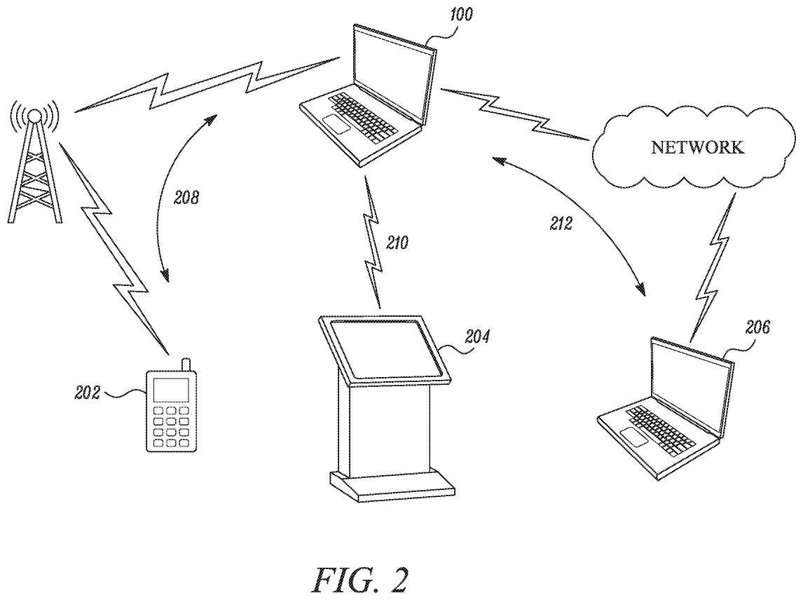

FIG. 2 illustrates a network environment 200 for implementation of the computing system 100, according to an embodiment of the present disclosure. As shown in FIG. 2, the computing system 100 is implemented as a remote device or a server. An individual or the doctor or the healthcare professional may communicate remotely with the computing system 100 though a portable device 202, a kiosk 204, or a desktop 206. The computing system 100 may communicate with the portable device 202 through wireless telecommunication interface represented as 208. Further, the computing system 100 may communicate with the kiosk 204 over wired interface 210. The computing system 100 may communicate with the desktop 206 over world wide web internet connection 212. These connections are for representational purpose and shall not limit the scope of communication in any manner. While several examples are described in the description, modifications, adaptations, and other implementations are possible. Accordingly, the following detailed description is not limited by the disclosed example. The processing unit 102 may be a centralized system (which may be implemented on a server or a cloud server, etc.) connected to the various other components of the computing system 100 via a network (not shown), such as internet or intranet, etc.

In an example implementation, with reference to FIG. 2, the portable device 202, the kiosk 204, or the desktop 206 may capture one or more images of the visual markers and/or other details of the individual for creating profile and transfer these details to the computing system 100. The computing system 100 may receive these details from the portable device 202, the kiosk 204, or the desktop 206 via network interface 110.

The computing system 100 may process the information received from the devices 202, 204, and 206 to determine disease type, constitution type, and health fingerprint associated with an individual and provides recommendations along with the details of the determined disease type, constitution type, and health fingerprint to the portable device 202, the kiosk 204, or the desktop 206.

Accordingly, the portable device 202 may be provided with an application installed therein to provide user interface for the operator of the portable device 202 to input details and to receive information displayed thereon, which may a determined health condition or recommendation. Similarly, the kiosk 204 and desktop 206 may have software installed thereon which allows the user of the kiosk 204 or the desktop 206 to submit images and other details. The said software may communicate these details to the computing system 100. In an example implementation, the kiosk 204 may function as a computing system 100.

In an example implementation, computing system 100 may be implemented as a server and the health condition determination is implemented as a service hosted by the computing system 100 over the internet. Thus, a user of the desktop 206 may browse the server to provide relevant details of the individual to determine health condition and receive recommendations.

The computing system 100 may implement the Artificial Intelligence to learn and develop correlations between different parameters/indicators and their corresponding disease, constitution, health fingerprint, and recommendations. Further, the computing system 100 may implement the Artificial Intelligence with data to learn mapping of different parameters/indicators with the details provided in the profile of an individual to determine underlying heath conditions.

In an example implementation, the computing system 100 may implement the Artificial Intelligence to analyze visual markers from the images and map them to causal plane for health fingerprint, disease progression, and healing support for display at the output device.

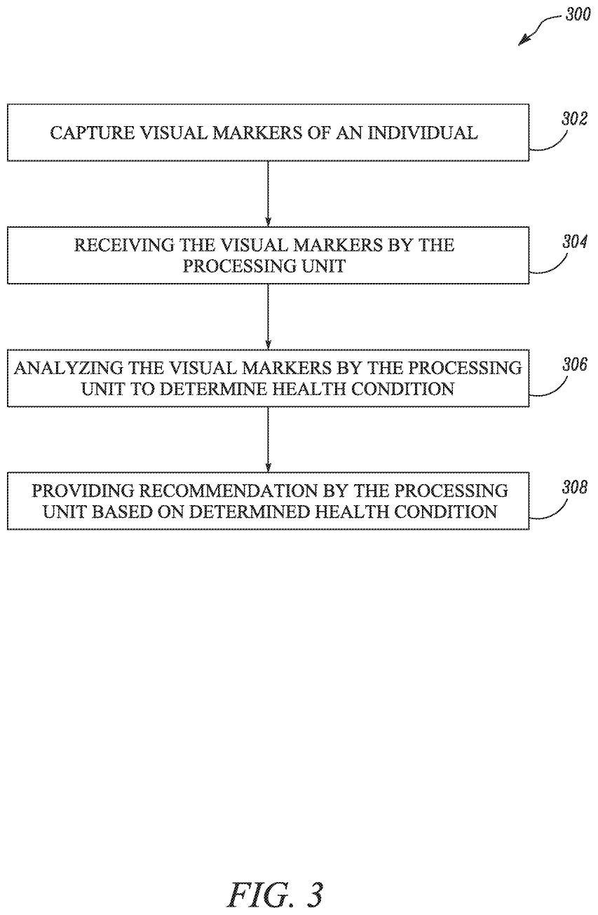

FIG. 3 illustrates a method 300 for determining a health condition of an individual based on an image of visual markers, according to an embodiment of the present disclosure. At step 302, at least an image of visual markers of the individual is captured by an imaging system. The image of visual markers includes images of face, hair, nails, and other body parts of the individual. At step 304, the captured image of visual markers is received by the processing unit 102. At step 306, the captured image of visual markers is analyzed by the processing unit 102 to determine the health condition of the individual. The determination of the health condition of an individual comprises determination of one or more of a constitution type, a disease type, a health fingerprint, a reactive allergy, symptoms of a disease, susceptibility to a disease, tendency to develop illness, a functional imbalance, and a prediction of a functional imbalance. The health condition may be indicative of susceptibility to diseases, root cause identification, disease progression, or health fingerprint.

In accordance with various embodiments of the present disclosure, the determination of the health condition is done by extracting one or more diagnostic parameters from at least an image of visual markers of the individual. Further, the processing unit 102 determines a correlation between the extracted one or more diagnostic parameters and a stored set of diagnostic parameters (in the first, second, and third databases) corresponding to each of the plurality of health conditions. The processing unit 102, upon determining the correlations, assigns weightages to the determined correlations based on the strengths of the correlations and determines the health condition based on the assigned weightages.

At step 308, one or more health recommendations are provided by the processing unit 102 based on the determined health condition. In an embodiment, the provided recommendations may be related to health supplements, medicines, medical support, exercise, diet management, lifestyle management, personalized body care products, wellness plan, precautions, additional health test, and sickness management.

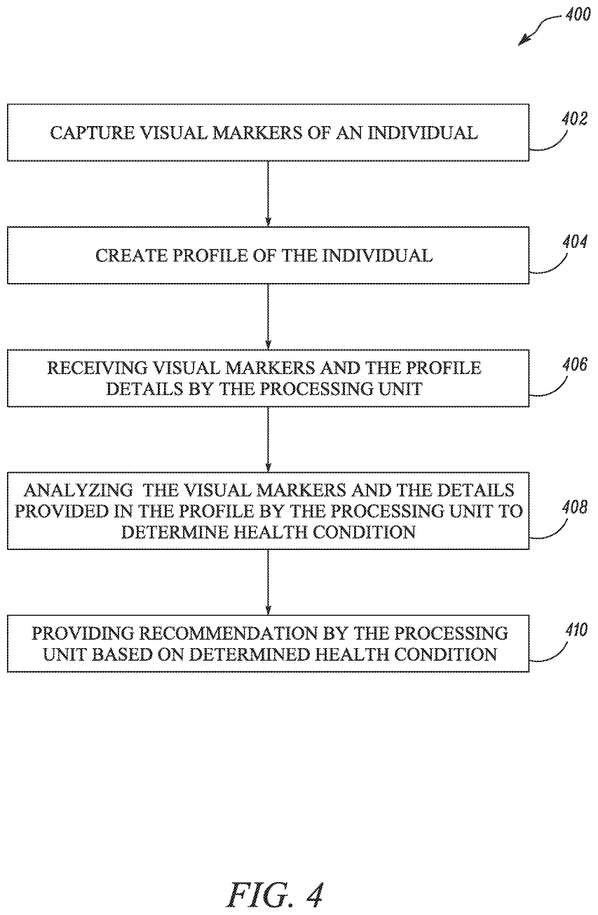

FIG. 4 illustrates a method 400 for determining a health condition of an individual based on at least an image of visual markers and other data, according to an embodiment of the present disclosure. At step 402, the image of visual markers of the individual is captured by an imaging system. The image of visual markers includes images of face, hair, nails, and other body parts of the individual. At step 404, a profile of the individual is created by the processing unit 102. The profile of the individual comprises one or more of name, address, age, height, weight, gender, physiological parameters, medical history, previous medical records, previous test reports, lifestyle details, diet intake, water intake, stress level, and occupational details of the individual.

At step 406, the captured image of visual markers along with the details from the individual's profile are received by the processing unit 102. At step 408, the visual marker and individual's profile are analyzed by the processing unit 102 to determine health condition. In an embodiment, the health condition of the individual may be first determined based on the image of the visual markers and then updated based on the individual's profile. The determination of the health condition of an individual comprises determination of one or more of a constitution type, a disease, a health fingerprint, a reactive allergy, symptoms of a disease, susceptibility to a disease, tendency to develop illness, a functional imbalance, and a prediction of a functional imbalance. The health condition may be indicative of susceptibility to diseases, root cause identification, disease progression, or health fingerprint.

In accordance with various embodiments of the present disclosure, the determination of the health condition is done by extracting one or more diagnostic parameters from the image of visual markers and the individual's profile. Further, the processing unit 102 determines a correlation between the extracted one or more diagnostic parameters and a stored set of diagnostic parameters (in the first, second, and third databases) corresponding to each of the plurality of health conditions. The processing unit 102, upon determining the correlations, assigns weightages to the determined correlations based on the strength of the correlations and determines the health condition based on the assigned weightages.

At step 410, one or more health recommendations are provided by the processing unit 102 based on the determined health condition. In an embodiment, the provided recommendations may be related to health supplements, medicines, medical support, exercise, diet management, lifestyle management, personalized body care products, wellness plan, precautions, additional health test, and sickness management.

With the systems and methods of present disclosure, the health condition of an individual may be determined remotely and in a non-invasive manner. Moreover, with the techniques implemented in the present disclosure, the health condition may be determined without need of any specific medical equipment and without any need to see a health practitioner. Since an image of an individual along with some basic profile data is utilized to determine the health condition of an individual, the process for determining the health condition of an individual become super easy and efficient.

Moreover, with the systems and methods of present disclosure, an efficient and highly accurate assessment of an individual health can be performed that considers various factors such as a sickness or a disease, reactive allergies, symptoms of a disease, susceptibility to a disease, root cause, constitution type, health fingerprint, tendency to develop illness, a functional imbalance, a prediction of a functional imbalance associated with the individual, and so on.

With the systems and methods of present disclosure, the accessibility and speed of care in the medical sector can be improved by providing diagnosis, treatment, and health recommendations faster and at an early stage. Also, since the determination of the health condition is simple and quick with the use of present disclosure, the throughput of the consultation with the medical professionals can be increased. Moreover, the systems and methods of the present disclosure enables the scaling in the medical sector by standardizing the quality of diagnosis, treatment, and recommendations provided to the patients.

A person having ordinary skills in the art will appreciate that the system, modules, and sub-modules have been illustrated and explained to serve as examples and should not be considered limiting in any manner. It will be further appreciated that the variants of the above disclosed system elements, or modules and other features and functions, or alternatives thereof, may be combined to create other different systems or applications.

Those skilled in the art will appreciate that any of the aforementioned steps and/or system modules may be suitably replaced, reordered, or removed, and additional steps and/or system modules may be inserted, depending on the needs of a particular application. In addition, the systems of the aforementioned embodiments may be implemented using a wide variety of suitable processes and system modules and is not limited to any particular computer hardware, software, middleware, firmware, microcode, or the like.

It may be contemplated that the database described herein may be internal and/or external database and may include data repositories, or other data sources. Although, there is a single database described in the present disclosure, it may be contemplated by a person ordinarily skilled in the art that multiple databases can be used, without deviating from the scope of the present disclosure. In some embodiments, the databases may be implemented using a relational database, such as Sybase, Oracle, CodeBase and Microsoft® SQL Server as well as other types of databases such as, for example, a flat file database, an entity-relationship database, and object-oriented database, a record-based database, or the like.

The claims can encompass embodiments for hardware, software, or a combination thereof. It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art, which are also intended to be encompassed by the following claims.

While aspects of the present disclosure have been particularly shown, and described with reference to the embodiments above, it will be understood by those skilled in the art that various additional embodiments may be contemplated by the modification of the disclosed machines, systems, and methods without departing from the spirit and scope of what is disclosed. Such embodiments should be understood to fall within the scope of the present disclosure as determined based upon the claims and any equivalents thereof.

We claim:

1. A method (300, 400) for determining a health condition of an individual, the method (300, 400) comprising:
receiving, by a processing unit (102), an image of visual markers of the individual; and
determining, by the processing unit (102), the health condition of the individual based on the image of visual markers of the individual, wherein determining the health condition of the individual comprises determining a constitution type of the individual.

2. The method (300, 400) as claimed in claim 1, further comprising:
receiving, by the processing unit (102), a profile of the individual; and
determining, by the processing unit (102), the health condition of the individual based on the profile of the individual;
providing, by the processing unit (102), one or more health recommendations based on the determined health condition of the individual.

3. The method (300, 400) as claimed in claim 1, wherein determining the health condition of the individual further comprises determining one or more of a disease, a health fingerprint, a reactive allergy, symptoms of a disease, susceptibility to a disease, tendency to develop illness, a functional imbalance, and a prediction of a functional imbalance.

4. The method (300, 400) as claimed in claim 1, wherein determination of the health condition of the individual comprises:
extracting, by the processing unit (102), one or more diagnostic parameters from the image of visual markers of the individual;
determining, by the processing unit (102), a correlation between the extracted one or more diagnostic parameters and a stored set of diagnostic parameters corresponding to a plurality of health conditions;
assigning, by the processing unit (102), weightages to the determined correlation based on a strength of correlation; and
determining, by the processing unit (102), the health condition based on the assigned weightages.

5. The method (300, 400) as claimed in claim 2,
wherein providing the one or more health recommendations comprises providing recommendations related to health supplements, medicines, medical support, exercise, diet management, lifestyle management, personalized body care products, wellness plan, precautions, additional health test, and sickness management, and
wherein the image of visual markers comprises an image of face, hair, or nail, of the individual.

6. A computing system (100) comprising:
a processing unit (102) configured to:
receive an image of visual markers of an individual; and
determine a health condition of the individual based on the image of visual markers of the individual, wherein determining the health condition of the individual comprises determining a constitution type of the individual.

7. The computing system (100) as claimed in claim 6, wherein the processing unit (102) is further configured to:
receive a profile of the individual; and
determine the health condition of the individual based on the profile of the individual; and
provide one or more health recommendations based on the determined health condition of the individual.

8. The computing system (100) as claimed in claim 6, wherein determining the health condition of the individual further comprises determining one or more of a disease, a health fingerprint, a reactive allergy, symptoms of a disease, susceptibility to a disease, tendency to develop illness, a functional imbalance, and a prediction of a functional imbalance.

9. The computing system (100) as claimed in claim 6, wherein the processing unit (102) is configured to determine the health condition of the individual by:
extracting one or more diagnostic parameters from the image of visual markers of the individual;
determining a correlation between the extracted one or more diagnostic parameters and a stored set of diagnostic parameters corresponding to a plurality of health conditions;
assigning weightages to the determined correlation based on a strength of correlation; and
determining the health condition based on the assigned weightages.

10. The computing system (100) as claimed in claim 7,
wherein providing the one or more health recommendations comprises providing recommendations related to health supplements, medicines, medical support, exercise, diet management, lifestyle management, personalized body care products, wellness plan, precautions, additional health test, and sickness management, and
wherein the image of visual markers comprises an image of face, hair, or nail, of the individual.

11. One or more computer-readable media having encoded thereon computer-executable instructions that, when executed by a computing system, cause the computing system to perform a method for determining a health condition of an individual, the method comprising:
receiving an image of visual markers of the individual; and
determining the health condition of the individual based on the image of visual markers of the individual, wherein determining the health condition of the individual comprises determining a constitution type of the individual.
